(19) United States
(12) Patent Application Publication
(10) Pub. No.: US 2015/0290160 A1
(43) Pub. Date: Oct. 15, 2015

(54) Spermidine/Spermine N1-Acetyltransferase Substrates As Anti-Cancer Drug Compounds

(71) Applicant: BIOMARK TECHNOLOGIES INC., Richmond (CA)

(72) Inventors: Brian Cheng, Chesterfield, MO (US); Rashid Bux, Vancouver (CA); Derek Cheng, Chesterfield, MO (US)

(21) Appl. No.: 14/443,050

(22) PCT Filed: Nov. 14, 2013
(86) PCT No.: PCT/CA2013/050873

§ 371 Date: May 14, 2015

Related U.S. Application Data
(60) Provisional application No. 61/726,534, filed on Nov. 14, 2012.

(51) Int. Cl. A61K 31/198; A61K 31/137; A61K 31/13

(52) U.S. Cl. CPC A61K 31/198; A61K 31/13; A61K 31/137

(57) ABSTRACT

An anti-cancer drug compound comprises an spermidine/spermine N-acetyltransferase substrate. The spermidine/spermine N-acetyltransferase substrate may be a monoamine. The spermidine/spermine N-acetyltransferase substrate may be amantadine, rimantadine, dopamine or L-DOPA. A method of treating cancer comprises the use of an spermidine/spermine N-acetyltransferase substrate to treat cancer. The spermidine/spermine N-acetyltransferase substrate may be a monoamine. The spermidine/spermine N-acetyltransferase substrate may be amantadine, rimantadine, dopamine or L-DOPA.

### BACKGROUND OF THE INVENTION

1. Field of the Invention

The present invention relates to a method for assaying the spermidine/spermine N¹-acetyltransferase (SSAT) activity of mRNA up-regulated cancer cells and, in particular, to the use of SSAT substrates as anti-cancer drug compounds and in anti-cancer treatments.

2. Description of the Related Art

U.S. Pat. No. 6,811,967 which issued to Sitar et al. on Nov. 4, 2004, and the full disclosure of which is incorporated herein by reference, discloses a method for assaying activity of the enzyme SSAT using SSAT substrates by detecting acetylated forms of the SSAT substrates. The SSAT substrates may include amantadine wherein metabolism of amantadine occurs in part by the action of the inducible enzyme SSAT to produce the acetylated metabolite N-acetylamantadine. Disclosed also is the correlation of SSAT activity to pathological conditions.

SSAT is an important enzyme in polyamine metabolism. Polyamines, including spermidine and spermine, are essential for cell survival and SSAT is a rate-limiting enzyme in the catabolic pathway which converts spermidine and spermine into acetylpolyamines to maintain intracellular polyamine homeostasis. It has been reported that in certain cancer cell lines a high expression of SSAT mRNA have been detected. See, for example, Chen et al. Genomic identification and biochemical characterization of a second spermidine/spermine N¹-acetyltransferase. Biochemical Journal. (2003), Volume 373, 661-667, the full disclosure of which is incorporated herein by reference.

It has also been reported that SSAT expression and enzymatic activity may be elevated following chemotherapy or treatment with spermidine analogues. In vitro cell line studies have further positively correlated SSAT expression and enzymatic activity with levels of cytotoxicity of new drug candidates. A number of anti-proliferative agents and polyamine analogues have accordingly been developed to prevent cancer cell proliferation via SSAT induction. See for example, Wallace, H. M. et al. A perspective of polyamine metabolism. Biochemical Journal. (2003), Volume 376, 1-14, the full disclosure of which is incorporated herein by reference.

### SUMMARY OF THE INVENTION

It is an object of the present invention to provide an improved anti-cancer drug compounds and anti-cancer treatment.

Certain cancer cells have high expressed spermidine/spermine N¹-acetyltransferase (SSAT) mRNA which can be treated with SSAT substrates to inhibit the acetylation of the polyamines by SSAT and catabolized biochemically. SSAT substrates are effective anti-cancer agents against the cancer cells with high expression of SSAT mRNA. The method allows a cancer type screening and identifies an effective anti-cancer drug treatment to enhance the cancer treatment efficacy. The method disclosed herein also allows for assaying the SSAT mRNA up-regulated cancer cells and use of SSAT substrates in anti-cancer treatments.

There is accordingly provided an anti-cancer drug compound comprising an SSAT substrate. The SSAT substrate may be a monoamine. The SSAT substrate may be amantadine, rimantadine, dopamine or L-DOPA. There is also provided a method comprising the use of an SSAT substrate to treat cancer. The SSAT substrate may be a monoamine. The SSAT substrate may be amantadine, rimantadine, dopamine or L-DOPA.

### BRIEF DESCRIPTIONS OF DRAWINGS

The invention will be more readily understood from the following description of the embodiments thereof given, by way of example only, with reference to the accompanying drawings, in which:

FIG. 1 shows the relative spermidine/spermine N¹-acetyltransferase (SSAT) expression levels by RT-qPCR assay and metabolic activities as measured by N-acetylspermidine formation in U2-OS, HeLa, Malme-3M, PC-3 and HEK293 human tumor cell lines;

FIG. 2 also shows the relative SSAT expression levels by RT-qPCR assay and metabolic activities as measured by N-acetylspermidine formation in U2-OS, HeLa, Malme-3M, PC-3 and HEK293 human tumor cell lines;

FIG. 3 shows the relative percent confluency of human tumor cell lines, U2-OS, HeLa, Malme-3M, PC-3 and HEK293 during incubation with spermidine from 22 μM to 550 μM;

FIG. 4 shows a summary of cytotoxicity and SSAT expression levels in human cell lines;

FIG. 5 shows a cytotoxic potential of monoamine test drugs amantadine, rimantadine, dopamine and L-DOPA, and a polyamine positive control spermidine against the human cancer cell line A549;

FIG. 6 shows a cytotoxic potential of monoamine test drugs amantadine, rimantadine, dopamine and L-DOPA, and a polyamine positive control spermidine against the human cancer cell line H322;

FIG. 7 shows a cytotoxic potential of monoamine test drugs amantadine, rimantadine, dopamine and L-DOPA, and a polyamine positive control spermidine against the human cancer cell line NCI-H23;

FIG. 8 shows a cytotoxic potential of monoamine test drugs amantadine, rimantadine, dopamine and L-DOPA, and a polyamine positive control spermidine against the human cancer cell line MCF-7;

FIG. 9 shows a cytotoxic potential of monoamine test drugs amantadine, rimantadine, dopamine and L-DOPA, and a polyamine positive control spermidine against the human cancer cell line T-47D;

FIG. 10 shows a cytotoxic potential of monoamine test drugs amantadine, rimantadine, dopamine and L-DOPA, and a polyamine positive control spermidine against the human cancer cell line BT-549;

FIG. 11 shows a cytotoxic potential of monoamine test drugs amantadine, rimantadine, dopamine and L-DOPA, and a polyamine positive control spermidine against the human cancer cell line LNCaP;

FIG. 12 shows a cytotoxic potential of monoamine test drugs amantadine, rimantadine, dopamine and L-DOPA, and a polyamine positive control spermidine against the human cancer cell line PC-3;

FIG. 13 shows a cytotoxic potential of monoamine test drugs amantadine, rimantadine, dopamine and L-DOPA, and a polyamine positive control spermidine against the human cancer cell line Du145;

FIG. 14 shows a cytotoxic potential of monoamine test drugs amantadine, rimantadine, dopamine and L-DOPA, and a polyamine positive control spermidine against the human cancer cell line U2-OS;

FIG. 15 shows SSAT expression levels in human cancer cell lines relative to A549 using GADPH as an internal reference;

FIG. 16 shows SSAT expression levels in human cancer cell lines relative to A549 using hPRT1 as an internal reference; and

FIG. 17 shows a correlation of test drug potency (1/IC₅₀) Against SSAT expression in ten human cancer cell lines.

### DESCRIPTIONS OF THE PREFERRED EMBODIMENTS

A method of using spermidine/spermine N¹-acetyltransferase (SSAT) substrates as anti-cancer drug compounds is disclosed herein.

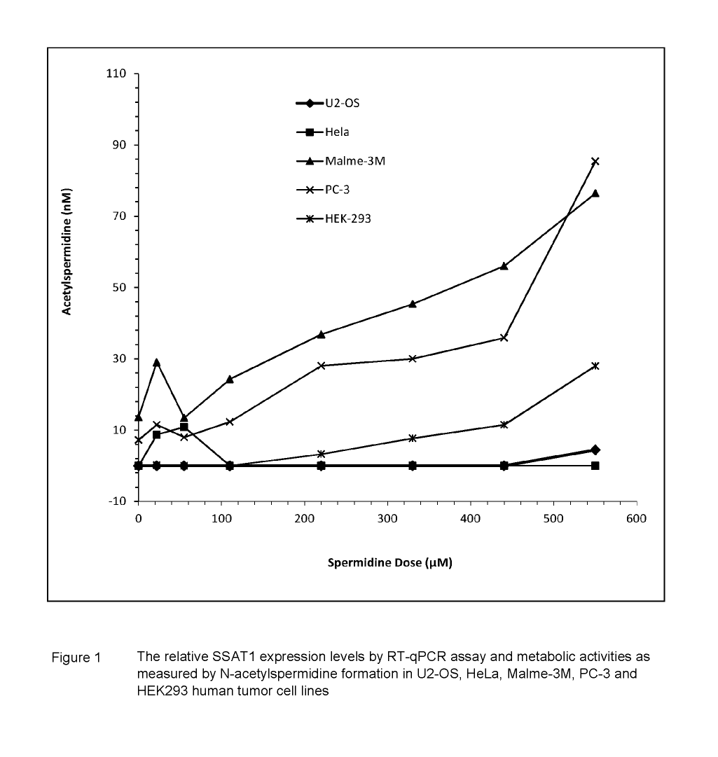

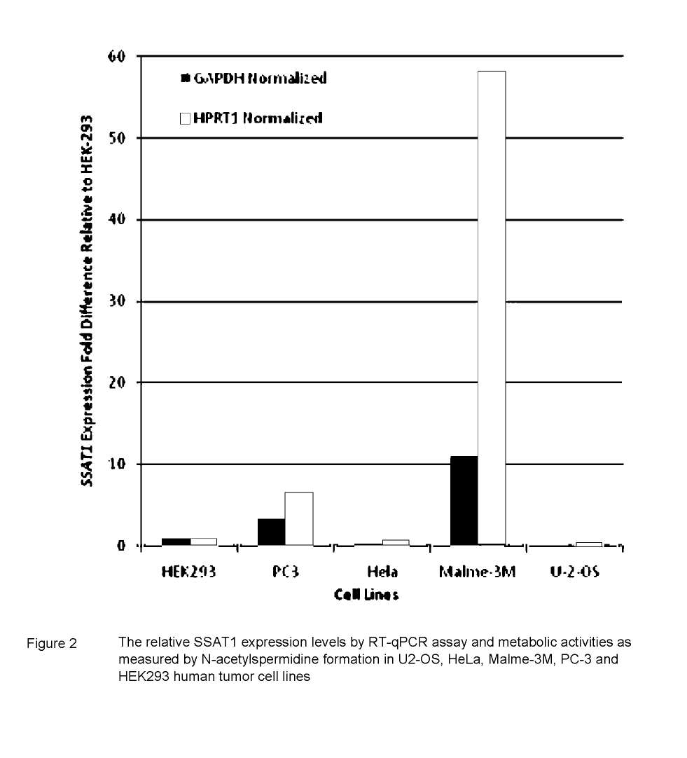

The relative SSAT expression levels in human tumor cell lines, HEK-293, Malme-3M, HeLa, PC-3 and US-02 cell lines were determined by a reverse transcription—quantitative polymerase chain reaction assay (RT-qPCR assay) and, as shown in FIGS. 1 and 2, Malme-3M was observed with the highest relative SSAT expression at 11-fold more than that of the control HEK-293 cell line when normalized with GAPDH and 58-fold more when normalized with HPRT1. PC-3 had the second highest expression level with approximately 3-fold and 7-fold differences of SSAT expression relative to HEK-293 when normalized with GAPDH and HPRT1, respectively. Both HeLa and U2-OS had lower SSAT expression levels than HEK-293. The SSAT expression levels were also compared against N-acetylated amantadine metabolite formation and the findings suggested a causal relationship between SSAT expression and N-acetylation metabolic activity.

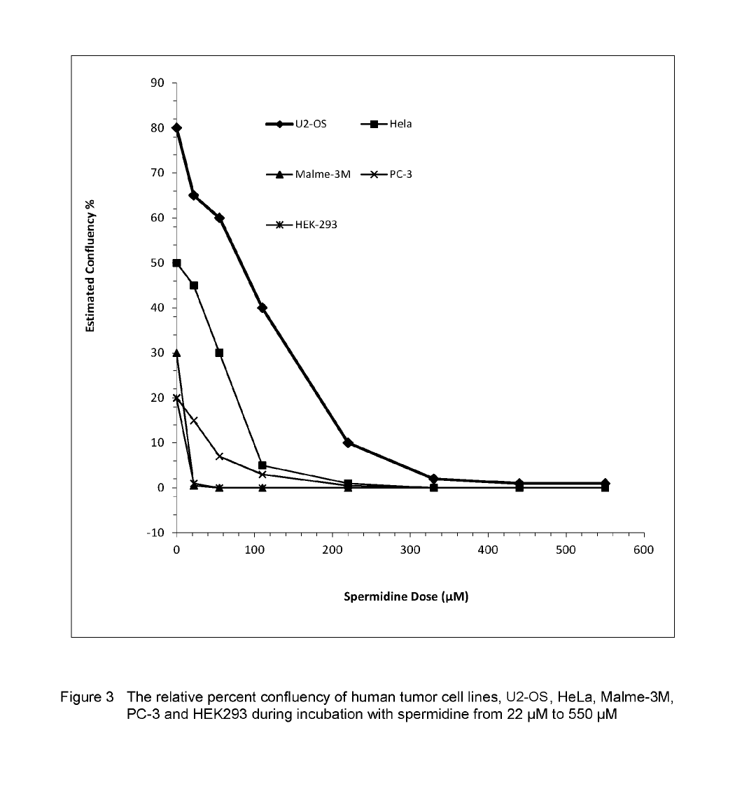

Referring now to FIG. 3, when the human tumor cell lines were incubated in the presence of spermidine from 22 μM to 550 μM, the relative cell viability, expressed as percent confluency, was observed to be highest in SSAT non-expressing cell lines (U2-OS and HeLa) with the lowest SSAT N-acetylation activity. In contrast, cell viability was observed to be lowest in SSAT over-expressing cell lines (Malme-M3 and PC-3). This data suggests that the significantly high cytotoxicity of spermidine in the human tumor cell lines is mediated by a metabolism-based mechanism of SSAT in tumors over-expressing SSAT.

It was subsequently shown that the SSAT substrates including amantadine, rimantadine, dopamine and L-DOPA will exhibit a selective and relative high level of cytotoxicity in human tumor cell lines over-expressing SSAT.

### Materials

### Monoamine Test Drugs

The following four monoamine test drugs were evaluated for cytotoxicity against human cancer cell lines.

Identity: Amantadine

| | |
|---|---|
| BRIVAL Reference No: | RFS-707 |
| Purity: | 99.0% |
| Batch/Lot No.: | 073K3695 |
| Supplier/Manufacturer: | Sigma |

Identity: Rimantadine

| | |
|---|---|
| BRIVAL Reference No: | ITS-31 |
| Purity: | 99.0% |
| Batch/Lot No.: | 07002MH |
| Supplier/Manufacturer: | Sigma |

Identity: Dopamine

| | |
|---|---|
| BRIVAL Reference No: | RFS-1016 |
| Purity: | 98.2% |
| Batch/Lot No.: | BCBB6599 |
| Supplier/Manufacturer: | Sigma |

Identity: L-DOPA

| | |
|---|---|
| BRIVAL Reference No: | RFS-982 |
| Purity: | 99.0% |
| Batch/Lot No.: | 099K1182 |
| Supplier/Manufacturer: | Sigma |

### Positive Control

Spermidine being a polyamine substrate for SSAT was used as a positive control test drug.

Identity: Spermidine

| | |
|---|---|
| BRIVAL Reference No: | RFS-1085 |
| Purity: | 99.8% |
| Batch/Lot No.: | 1441607 |
| Supplier/Manufacturer: | Sigma |

### Human Cell Lines

Three cell lines from each of lung, breast and prostate cancers have been selected based on literature SSAT expression data and used for potential cytotoxicity screening in the MTT assay. The SSAT non-expressing human osteosarcoma cell line U2-OS was used as the negative control cell line. The following human cell lines were used:

| | ATCC | |
|---|---|---|
| Cell Line Designation | Number¹ | Cancer Type |
| A549 | CCL-185 | Lung Carcinoma |
| H322 | n/av | |
| NCI-H23 | CRL-5800 | |
| MCF-7 | HTB-22 | Breast Cancer |
| T-47D | HTB-133 | |
| BT-549 | HTB-122 | |
| LNCaP | CRL-1740 | Prostate Carcinoma |
| PC-3 | CRL-1435 | |
| Du-145 | HTB-81 | |
| U2-OS | HTB-96 | Osteosarcoma |

¹American Type Culture Collection

### Experimental Procedures

Each human cancer cell line was incubated with each of the four monoamine test drugs at a range of testing concentrations. Cytotoxicity expressed as half maximal inhibitory concentration or IC₅₀ was determined based on a (dimethyl-2-thiazolyl)-2,5-diphenyl-2H-tetrazolium bromide or MTT assay. In parallel, the expression levels of SSAT in these cell lines were measured using a RT-qPCR assay.

### Preparation of Human Cell Lines

Cells of the ten cell lines were harvested from their established adherent cultures with trypsin EDTA, pelleted by centrifugation, and resuspended in the appropriate medium to yield a suspension of cells for each cell line.

### Evaluation of IC₅₀ by MTT Cytotoxicity Assay

Each of the monoamine test drugs (amantadine, rimantadine, dopamine and L-DOPA) and the positive control (spermidine) was accurately weighed, dissolved and further diluted with sterile water into a series of solutions at 100× of their target incubation concentrations. The target incubation concentrations for amantadine, rimantadine, and dopamine were 0.03, 0.1, 0.3, 1, 3, 10, 30, 100, 300 and 1000 μM. The target incubation concentrations for L-DOPA and the positive control, spermidine, were 0.1, 0.3, 1, 3, 10, 30, 100, 300 and 1000 μM.

Incubation was performed in triplicate at each drug concentration for each cell line tested. In parallel to each test drug treatment, each cell line was also treated with spermidine as positive controls.

An aliquot of the cell suspension was added into each well of 96-well culture plates and the plates were incubated overnight at 37° C. with a highly humidified atmosphere of 95% air and 5% carbon. On the following day each well was replaced with fresh medium and an aliquot of the appropriate test drug solution was added at 1% of the cell culture volume to achieve the target testing concentration. Blank culture media were used in lieu of the substrate solutions to prepare the vehicle controls (i.e. 0 μM substrate). The plates were then returned to incubation for three days at 37° C. with a highly humidified atmosphere of 95% air and 5% carbon.

After three days of incubation with substrates, an aliquot of 5 mg/mL MTT was added to each well and then incubated for 1 to 3 hours. Following incubation the medium was replaced with DMSO to dissolve the formazan. An aliquot from each well was measured for absorbance at 550 nm or 555 nm on a 96-well flat bottom plate with a microplate reader and DMSO for background absorbance correction.

### Examination of SSAT Expression Level by RT-qPCR

RNA extraction was performed using a QIAshredder™ Kit and RNeasy™ Mini Kit both which are available from Qiagen, Inc. having an address at Suite 200-27220 Turnberry Lane, Valencia, Calif., United States of America. Re-suspended cells from each of the cell lines tested were lysed with RNeasy™ lysis buffer. RNA was then extracted from the lysate using RNeasy™ Mini Spin columns. Sufficient total RNA concentration in each extracted sample was confirmed by Nanodrop spectrophotometric measurement.

RT-qPCR for SSAT was performed using a QuantiTect SYBR Green RT-PCR Kit also available from Qiagen, Inc. The reaction mixture for each sample consisted of QuantiTect SYBR Green RT-PCR master mix, QuantiTect RT mix, RNase-free water, SSAT PCR primers, and the extracted RNA from each sample.

Expression levels of the house-keeping genes GAPDH and HPRT1 were measured in parallel for each sample as outlined above using the corresponding PCR primers for these genes. SSAT expression levels were normalized with GAPDH or HPRT1 as the internal reference, and expressed as fold difference relative to that of A549.

### Instrumentation and RT-qPCR Program:

Instrument: Applied Biosystems Cycler
Reverse transcription: 20 min at 50° C.; 20° C./sec
Initial step: 15 min at 95° C.; 20° C./sec
   Cycling step:

Denaturation: 15 sec at 94° C.; 20° C./sec

Annealing: 20 sec at 55° C.; 20° C./sec

Extension: 30 sec at 72° C.; 20° C./sec
Number of cycles: 40

### Results and Discussion

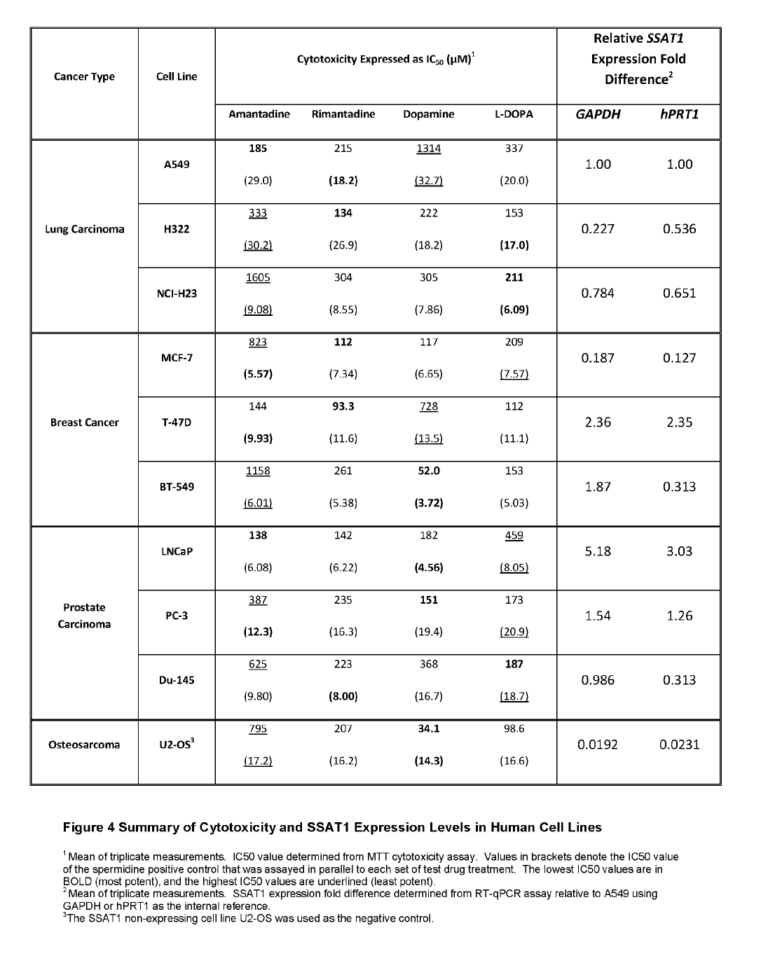

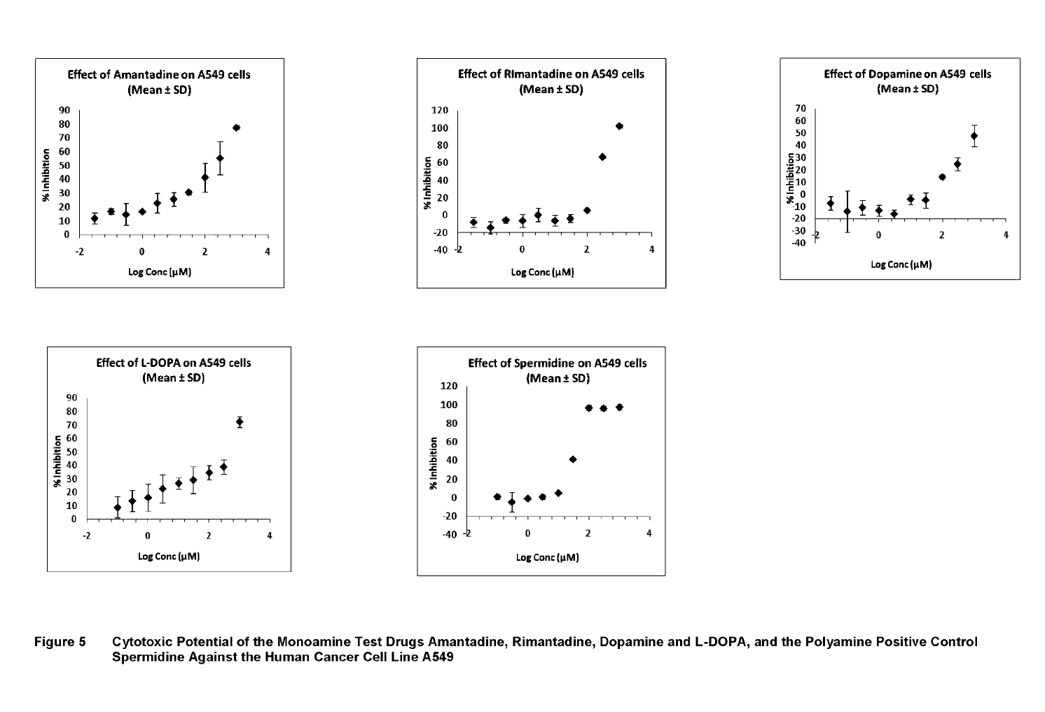

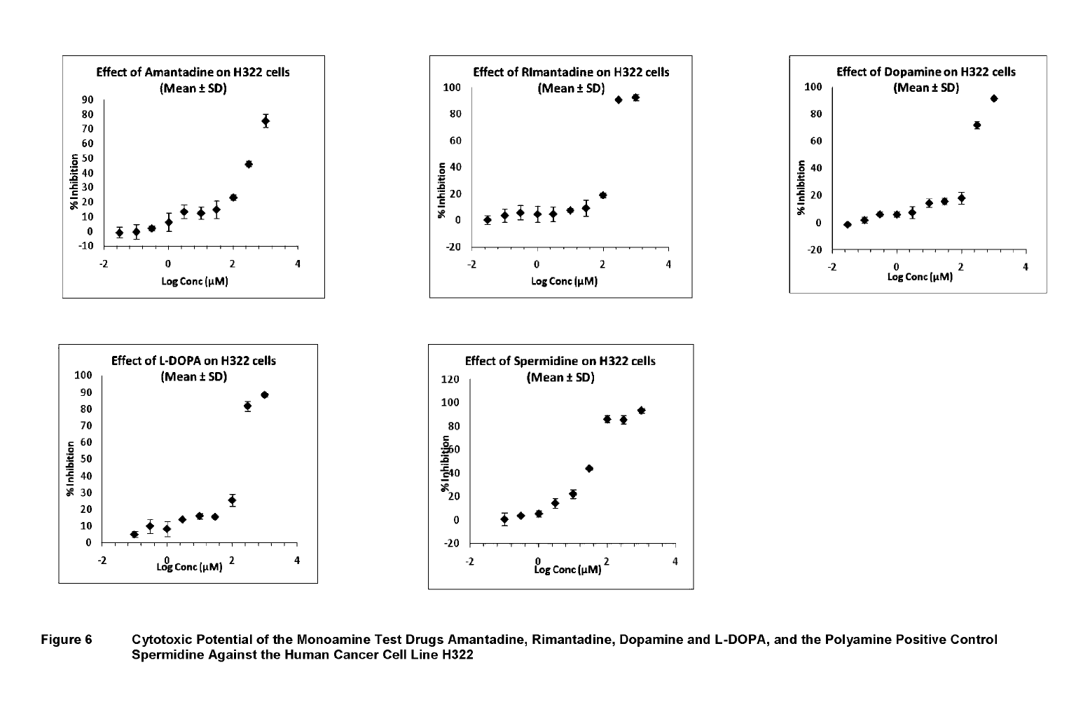

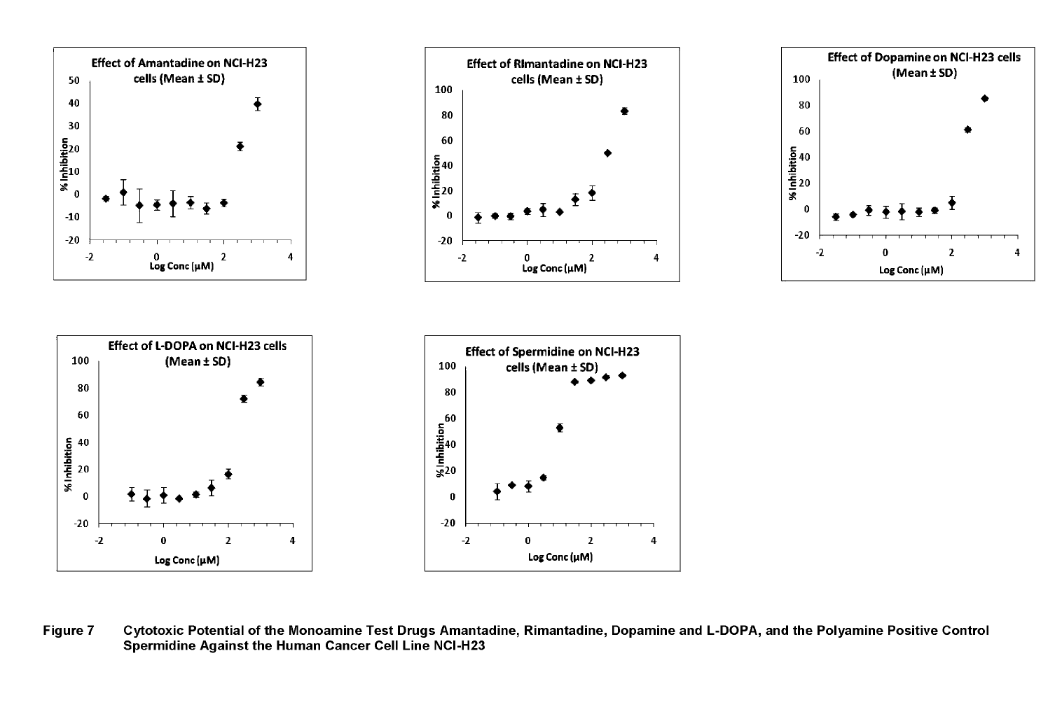

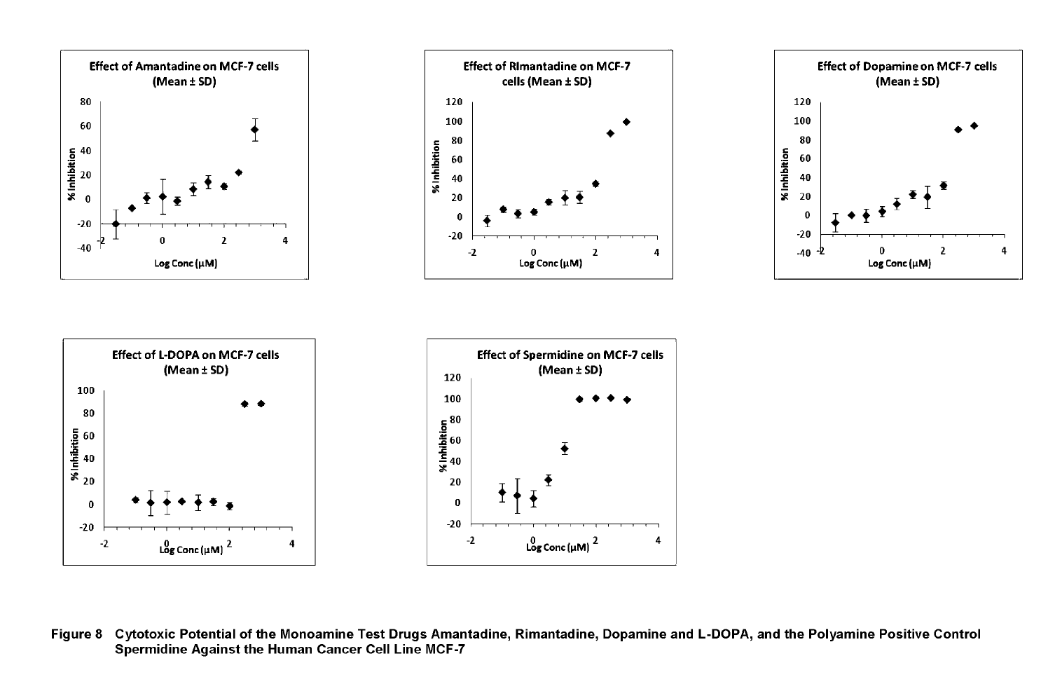

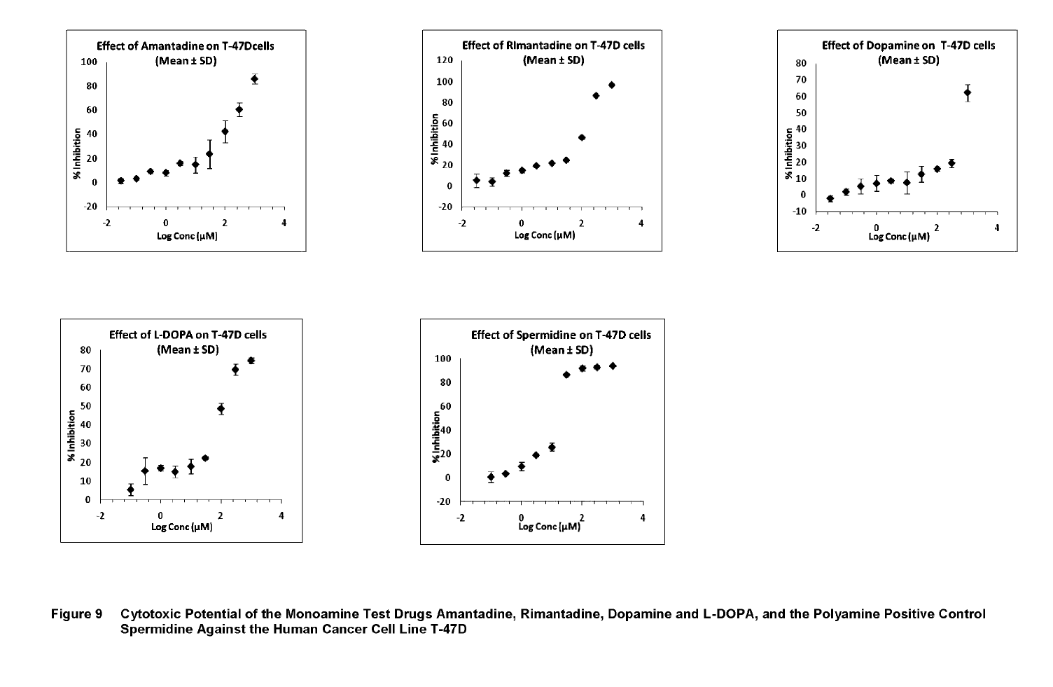

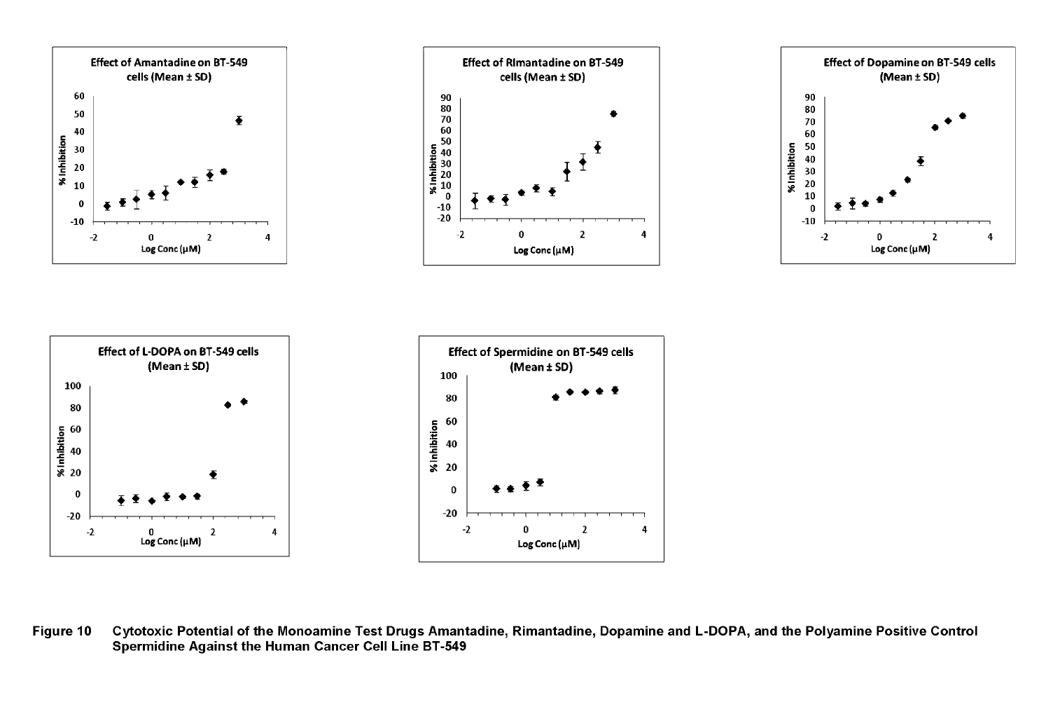

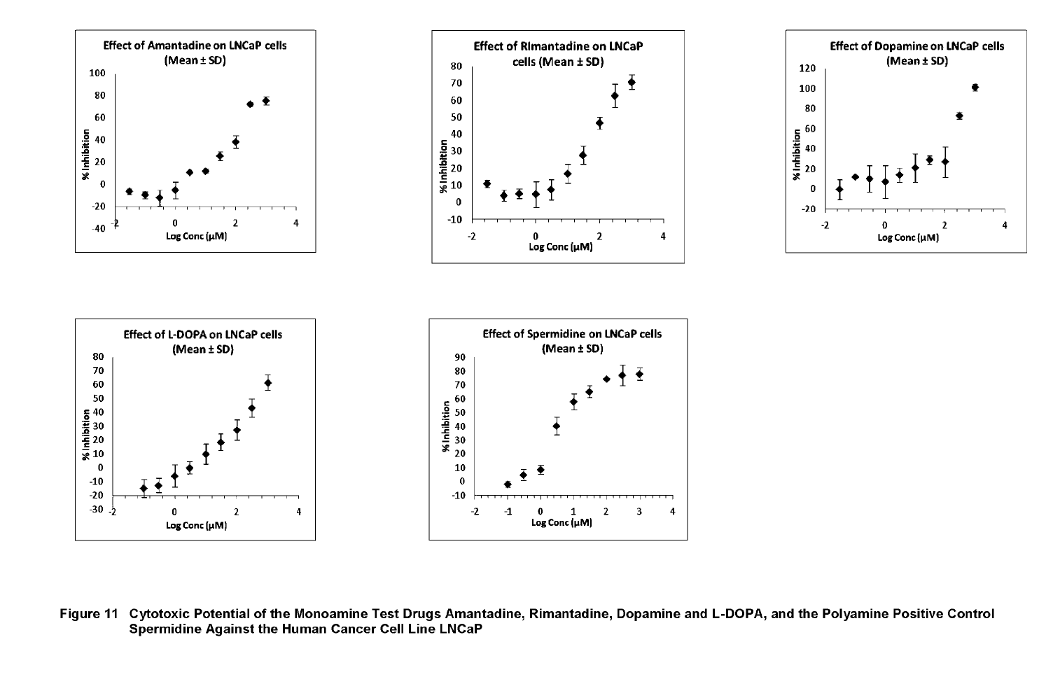

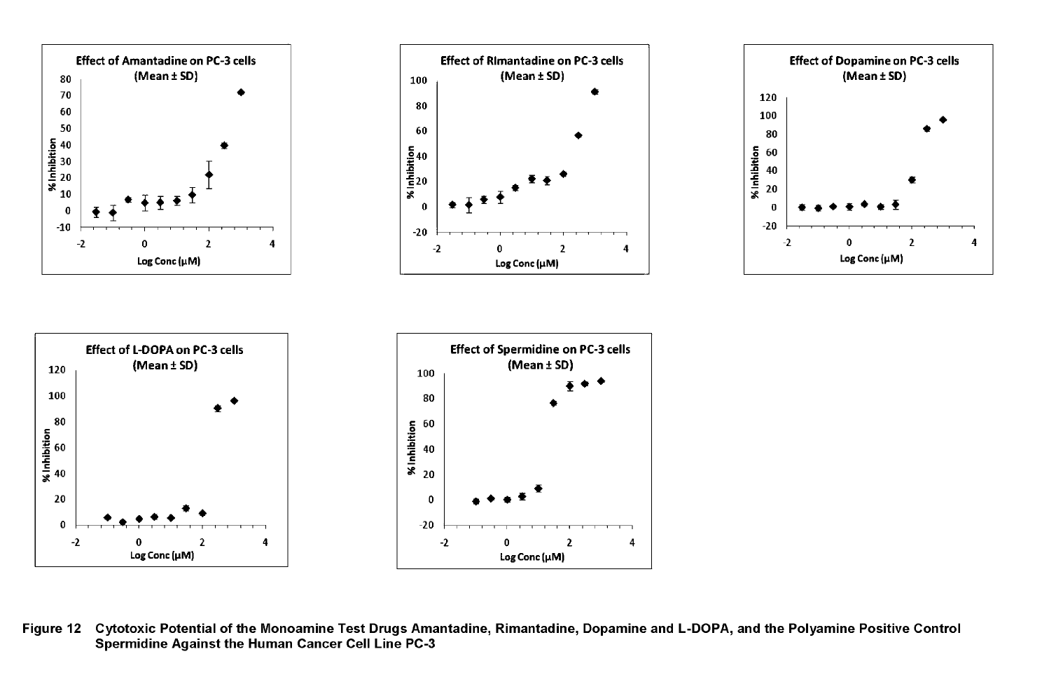

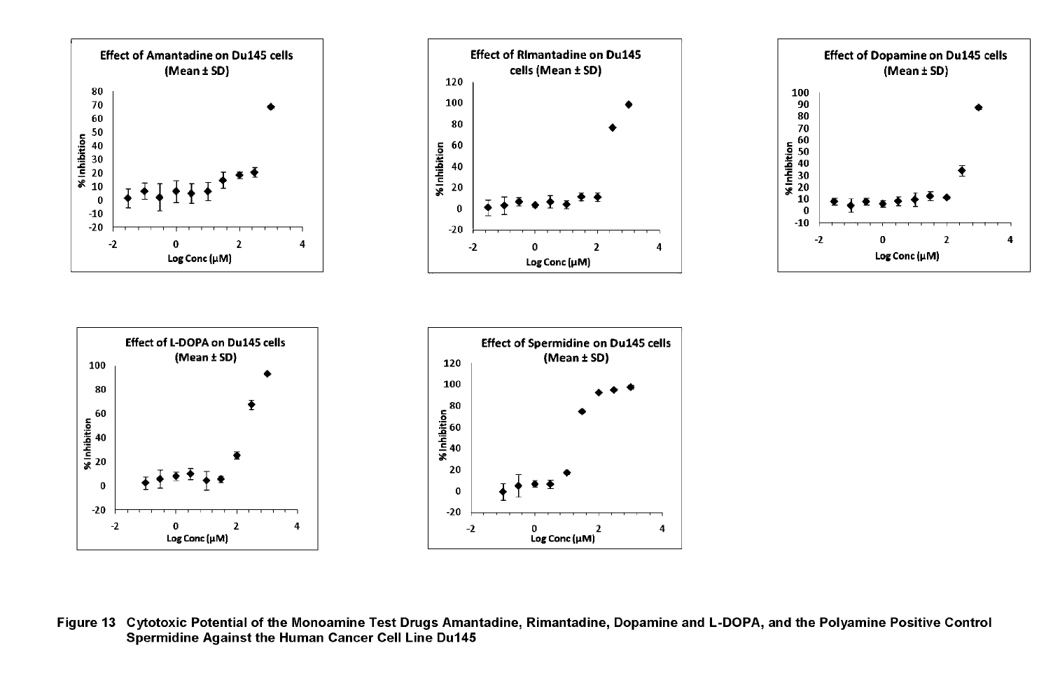

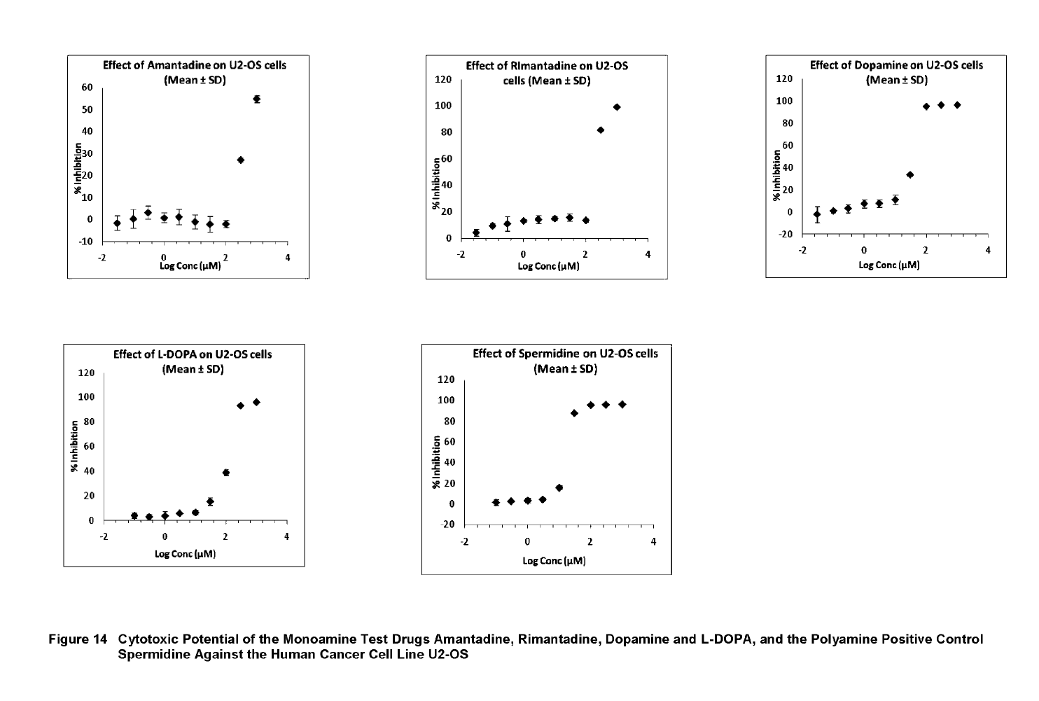

A summary of the cytotoxicity expressed as IC₅₀ for each SSAT monoamine test drug for each human cancer cell line tested is presented in FIG. 4. Cytotoxicity was determined based on treating each cell line with each of the test drugs over a range of concentrations. Following a three day incubation period cytotoxicity was measured by an MTT assay. IC₅₀ values were deduced based on plots of cytotoxicity level expressed as percentage inhibition over the testing concentrations as shown in FIGS. 5 to 14. In parallel to each drug treatment, each cell line was also treated with spermidine as a positive control and the IC₅₀ value for spermidine was determined for comparison.

From the data, the IC₅₀ values of the four monoamine test drugs ranged from 34.1 μM to 1605 μM across all the cell lines evaluated, with a majority between 100 μM and 500 μM. Overall, the most potent monoamine test drug was dopamine acting on the negative control U2-OS osteosarcoma cell line with an IC₅₀ value of 34.1 μM. This is followed by dopamine acting on the breast cancer cell line BT-549 with an IC₅₀ value of 52.0 μM. The least potent test drug was amantadine with IC₅₀ values of 1605 μM and 1158 μM when acting on NCI-H23 (lung cancer) and BT-549 (breast cancer), respectively.

The IC₅₀ values of the four monoamine test drugs were relatively higher than the positive control spermidine, ranging from 3.72 μM to 32.7 μM, reflecting that the monoamine test drugs were lower in cytotoxic potency compared with polyamine spermidine. For each cell line, it was noted that in general the rank-ordering of IC₅₀ (i.e. cytotoxicity) of the monoamine test drugs appeared to correlate with that of the polyamine spermidine positive control. This apparently similar rank-order of cytotoxicity may suggest a common mode of mechanism between the monoamine and polyamine drugs.

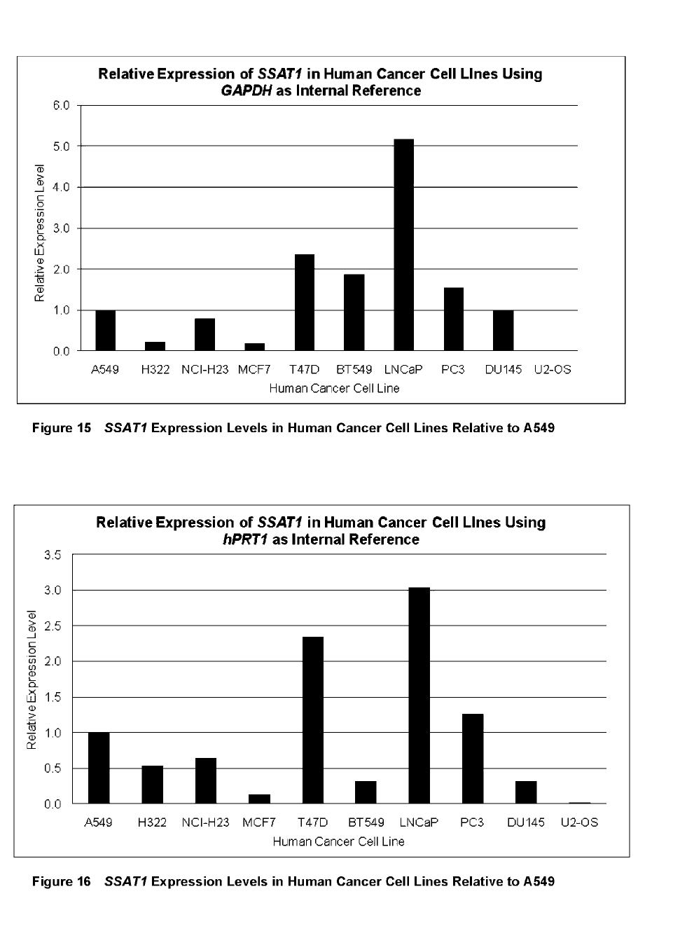

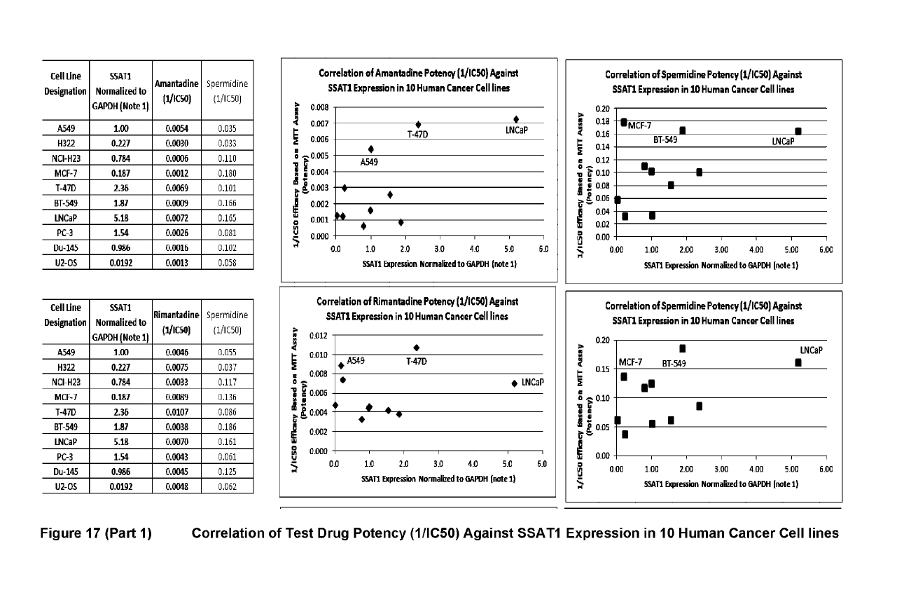

A summary of the relative SSAT expression levels in the human cell lines tested is presented in FIGS. 4, 15 and 16. The relative SSAT expression levels in the cell lines tested were examined based on a RT-qPCR assay. The cycle threshold of SSAT measured from each sample was normalized against that of the house-keeping gene GAPDH or HPRT1 as the internal reference to correct for potential variation in the amount and quality of RNA between the different samples. The results were then expressed as fold difference of SSAT expression level relative to the expression level in A549.

From the results, LNCaP was observed to have the highest relative expression level of SSAT with approximately 5-fold more than that of A549 when normalized with GAPDH, and 3-fold more when normalized with HPRT1. T-47D had the second highest expression level with approximately 2-fold difference of SSAT expression relative to A549. The SSAT non-expressing cell line U2-OS (negative control) had the lowest SSAT expression level as anticipated.

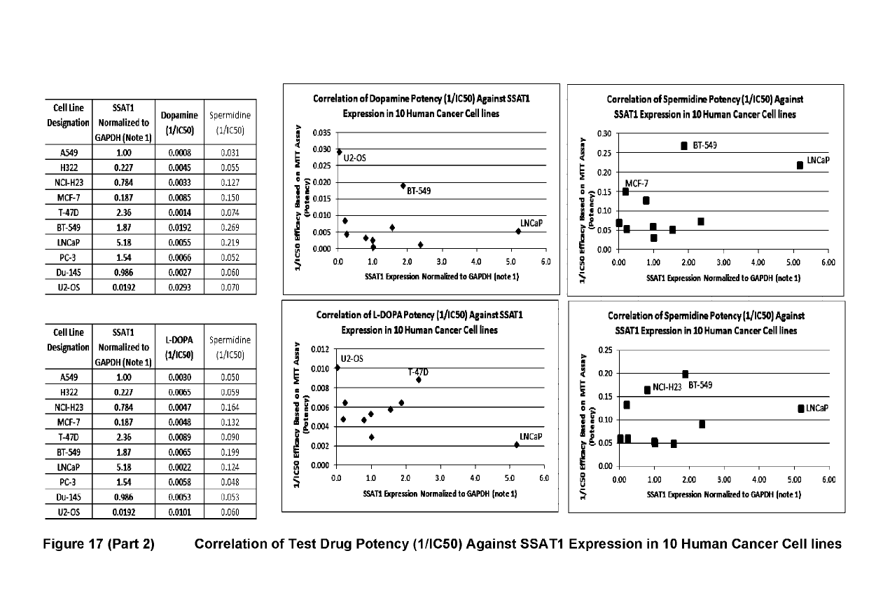

RT-qPCR results were compared against the IC₅₀ values of each test drug for each cell line in an attempt to correlate SSAT expression level with cytotoxicity of the monoamine test drugs. From correlation of SSAT expression against potency expressed as 1/IC₅₀, shown in FIG. 17, it is interesting to note that high SSAT expression is generally observed to be associated with high cytotoxicity potency, however, low SSAT expression was observed with high cytotoxicity for selected tumor cell lines. Cell lines that over-express SSAT, such as LNCaP (>5-fold change) and T-47D (>2 fold change), demonstrated high cytotoxicity (low IC₅₀) when treated with amantadine and rimantadine, but cytotoxicity is not consistently observed when treated with other monoamine drugs. In contrast, the negative control cell line, U2-OS, which does not express SSAT, displayed low IC₅₀ values (high cytotoxicity) when treated with dopamine and L-DOPA. The results therefore suggest that competitive inhibition of SSAT by selected monoamine test drugs appears to be effective only on certain tumors operating with a SSAT sensitive phenotype. A SSAT non-sensitive tumor phenotype was also noted amongst the 10 human tumor cell lines evaluated.

The monoamine test drugs amantadine, rimantadine, dopamine and L-DOPA were evaluated for cytotoxicity against three SSAT over-expressing human cancer cell lines from each of lung, breast and prostate cancers. Across all nine tumor cell lines tested, the cytotoxic potency of the monoamine test drugs were observed to be lower compared with spermidine which was a polyamine positive control. In general, the rank-ordering of cytotoxicity of the monoamine test drugs appeared to correlate with that of the polyamine spermidine, suggesting a common mode of mechanism between the monoamine and polyamine drugs. It is accordingly concluded that the monoamine test drugs and other SSAT substrates may be used as anti-cancer drug compounds and in anti-cancer treatment.

It will be understood by a person skilled in the art that many of the details provided above are by way of example only, and are not intended to limit the scope of the invention which is to be determined with reference to the following claims.

1. An anti-cancer drug compound comprising a spermidine/spermine N¹-acetyltransferase substrate.

2. The anti-cancer drug compound as claimed in claim 1 wherein the spermidine/spermine N¹acetyltransferase substrate is a monoamine.

3. The anti-cancer drug compound as claimed in claim 1 wherein the spermidine/spermine N¹acetyltransferase substrate is amantadine.

4. The anti-cancer drug compound as claimed in claim 1 wherein the spermidine/spermine N¹acetyltransferase substrate is rimantadine.

5. The anti-cancer drug compound as claimed in claim 1 wherein the spermidine/spermine N¹acetyltransferase substrate is dopamine.

6. The anti-cancer drug compound as claimed in claim 1 wherein the spermidine/spermine N¹acetyltransferase substrate is L-DOPA.

7. Use of a spermidine/spermine N¹acetyltransferase substrate to treat cancer.

8. Use of a spermidine/spermine N¹acetyltransferase substrate as claimed in claim 7 wherein the spermidine/spermine N¹acetyltransferase substrate is a monoamine.

9. Use of a spermidine/spermine N¹acetyltransferase substrate as claimed in claim 7 wherein the spermidine/spermine N¹acetyltransferase substrate is amantadine.

10. Use of a spermidine/spermine N¹acetyltransferase substrate as claimed in claim 7 wherein the spermidine/spermine N¹acetyltransferase substrate is rimantadine.

11. Use of a spermidine/spermine N¹acetyltransferase substrate as claimed in claim 7 wherein the spermidine/spermine N¹acetyltransferase substrate is dopamine.

12. Use of a spermidine/spermine N¹acetyltransferase substrate as claimed in claim 7 wherein the spermidine/spermine N¹acetyltransferase substrate is L-DOPA.
